(19) United States
(12) Patent Application Publication
(10) Pub. No.: US 2011/0177016 A1
(43) Pub. Date: Jul. 21, 2011

(54) Long Wear Cosmetic Composition

(75) Inventors: Jennifer Aliano, Annandale, NJ (US); Joseph F. Calello, Bridgewater, NJ (US); Christopher C. Frankfurt, Union Beach, NJ (US); Anjali A. Patil, Westfield, NJ (US); Julio G. Russ, Westfield, NJ (US); Ann M. Ureneck, Red Bank, NJ (US)

(73) Assignee: Revion Consumer Products, New York, NY (US)

(21) Appl. No.: 12/688,063

(22) Filed: Jan. 15, 2010

(51) Int. Cl. A61K 8/81; A61Q 1/10

(52) U.S. Cl. 424/70.7; 424/78.03

(57) ABSTRACT

The present invention is directed to a cosmetic composition containing at least one styrene/acrylate copolymer and retaining gloss properties, comfort without irritation, and staying power for up to about 8 hours.

This application claims priority from PCT application International Application Number PCT/US2008/073227, filed Aug. 15, 2008, claiming priority for U.S. provisional application No. 60/956,410, filed Aug. 17, 2007, now expired and the entire disclosure of which is hereby incorporated by reference.

The present invention relates to a composition containing at least one styrene/acrylate copolymer, intended for a cosmetic preparation.

The cosmetic composition may be used on the lips, eyelashes, eyebrows, hair, skin, and nails.

There are numerous cosmetic compositions for which the gloss properties of the deposited film, following application to the skin, lips, eyelashes, eyebrows, nails, and hair are very important. The compositions for example can be in the form of a mascara, eyeliner, lip product, face powder, or an eyeshadow.

The present disclosure relates to a composition for topical application comprising, in a cosmetically acceptable support, at least one styrene/acrylate copolymer, at least one siloxane, at least one rheology agent, and at least one ester. The composition may optionally contain at least one hydrocarbon, at least one particulate matter, at least one pigment, and at least one additive. This composition exhibits good cosmetic properties, for example up to 8 hours of staying power, gloss, and comfort without irritation. It has been found that the composition of this application retains gloss properties and does not need to be reapplied for up to about 8 hours.

The disclosed cosmetic composition comprises, by weight of the total composition:

a. about 3-35% of a styrene/acrylate copolymer;
b. about 0.1-20% of a rheology agent;
c. about 1-35% of a siloxane; and
d. about 1-35% of an ester.

The average gloss property may be measured with the aide of a gloss meter reading at an angle of between 20-60°. The gloss property is determined by comparing a test product against a base-coat of a traditional two step long-wearing lip product at time intervals between about 0.5 hours and about 10 hours.

“At least one” compound means one or more compounds.

Ester, as used herein, refers to the group —OC(O)C₁-C₂₀ straight chain alkyl. Providing the ester is not a C₈-C₁₆ branched ester. Examples of esters include; polyglyceryl-4 isostearate, hexyl laurate, trioctyldodecylcitrate and mixtures thereof.

The styrene/acrylate copolymer is derived from the polymerization of:
a) 10 to 55% by weight, relative to the total weight of monomers, of para-tert-butylstyrene,
b) 20 to 80% by weight C₁-C₁₀ alkyl methacrylate monomer,
c) 2 to 25% by weight C₁-C₁₀ alkyl acrylate monomer,
d) from 0 to 40% by weight of vinylaromatic monomer other than para-tert-butylstyrene.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups.

The vinyl aromatic monomer present in the copolymer can be selected from those containing 8 to 20 carbon atoms and are for example 2-methylstyrene, 3-methylstyrene, 4-methylstyrene, 4-propylstyrene, 4-cyclohexylstyrene, 4-dodecylstyrene, 2-ethyl-4-benzylstyrene, styrene, para-methylstyrene, ortho-methylstyrene and mixtures thereof.

The copolymer can be crosslinked and can thus comprise, in a known manner, at least one ethylenic crosslinking monomer. Crosslinking monomers include for example, ethylene glycol, dimethacrylate, ethylene glycol, diacrylate and divinylbenzene.

The crosslinking monomer can be present in the copolymer in an amount ranging from 0.1 to 10% by weight relative to the total weight of the mixture of monomers in the copolymer.

Styrene/acrylate copolymer comprises a tert-butylstyrene/C₁-C₁₀ alkyl acrylate/methacrylate copolymer.

As used herein, “about” shall generally mean within 20 percent.

A “non-volatile” compound is a compound capable of remaining on the skin or lips for a number of hours. A non-volatile compound has, for example, a non-zero vapor pressure, at ambient temperature and atmospheric pressure of less than 0.05 mm Hg (2.66 Pa).

A “hydrocarbon” compound is a compound comprising principally atoms of carbon and hydrogen and optionally one or more functional groups chosen from hydroxyl, ester, ether and carboxyl functions. Examples of a hydrocarbons includes isododecane (2,2,4,4,6-pentamethylheprane), isoparaffin, cyclomethicones, and linear methicones. the hydrocarbon can be present in the composition from about 3-40% by weight of the total composition.

Rheology agents include but are not limited to polymeric emulsifiers, thickening agents, synthetic and natural gum or polymer products, polysaccharide thickening agents, stabilizers, associative thickeners, anionic associative rheology modifiers, nonionic associative rheology modifiers, oil-thickening agents, natural and synthetic wax, 12-hydroxysteric acid, clays, silica, and trihydroxystearin, and combinations thereof.

The particulate phase present in the composition according to the present disclosure may comprise pigments and nacres and/or fillers commonly used in cosmetic compositions.

Pigments are to be understood as particles, which are dispersable in the fatty substances such as oils and which are intended for coloring and/or opacifying the composition. The pigments may be white or colored, mineral and/or organic, coated or uncoated, and spherical or oblong.

The composition according to the present disclosure may be in the form of a lip makeup product, such as lip-gloss.

The composition according to the present disclosure may be in the form of an eye makeup product such as mascara.

Siloxane is a organosilicon containing silicon, oxygen and an alkane. Examples include phenyl trimethicone, polysilicone-6, trimethylsiloxyphenyl dimethicone, and mixtures thereof.

The composition of the present disclosure may further comprise at least one additive commonly used in the cosmetic compositions, for example an additive can be water, dyes, aromas, fragrances, antioxidants, preservatives, neutralizing agents, aqueous-phase gelling agents, dispersants, botanical and seed extracts, cosmetic actives and mixtures thereof.

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods of the invention and how to make and use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. The use of examples anywhere in this specification, including examples of any terms discussed herein, is illustrative only, and in no way limits the scope and meaning of the invention or of any exemplified term. Likewise, the invention is not limited to the examples presented.

### EXAMPLE 1

A long wear lip-gloss was made as follows:

| Compound | w/w/% |
|---|---|
| Sequence 1 | |
| Styrene Acrylic Copolymer | 19.5 |
| Phenyl Trimethicone | 12 |
| Isododecane | 10 |
| Polyglyceryl-4 Isostearate, Cetyl | 9.57 |
| PEG/PPG-10/1 Dimethicone, Hexyl Laurate | |
| Silica silylate grind | 21 |
| Sequence 2 | |
| PPG-51/SMDI Copolymer | 8 |
| Trimethylsiloxyphenyl dimethicone | 10 |
| Microcrystalline rheology agent | 2.0 |
| 1,2 Hexanediol, caprylyl glycol | 0.5 |
| Sequence 3 | |
| Orange 5 | 0.05 |
| Organic or Inorganic Dry Color pigments | 1.58 |
| Sequence 4 | |
| Mica, Titanium Dioxide | 0.6 |
| Calcium Sodium Borosilicate Titanium | 0.50 |
| Dioxide | |
| Sequence 5 | |
| Mica | 4.00 |
| Fragrance | 0.4 |

Mix sequence 2 into sequence 1 in a steam bath. Add sequence 3 and mix until uniform. Add sequence 4 and mix until uniform. Add sequence 5 and mix until uniform. Add fragrance.

### EXAMPLE 2

A long wear lip-gloss was made as follows:

| Compound | w/w/% |
|---|---|
| Sequence 1 | |
| Styrene Acrylic Copolymer | 19.5 |
| Phenyl Trimethicone | 10 |
| Isododecane | 10 |
| Polyglyceryl-4 Isostearate, Cetyl | 16.72 |
| PEG/PPG-10/1 Dimethicone, Hexyl Laurate | |
| Silica silylate grind | 14 |
| Sequence 2 | |
| PPG-51/SMDI Copolymer | 10 |
| Trimethylsiloxyphenyl dimethicone | 10 |
| Microcrystalline rheology agent | 2.0 |
| 1,2 Hexanediol, caprylyl glycol | 0.5 |
| Sequence 3 | |
| Orange 5 | 0.05 |
| Organic or Inorganic Dry Color pigments | 1.46 |
| Sequence 4 | |
| Mica, Titanium Dioxide | 0.5 |
| Calcium Sodium Borosilicate Titanium | 0.50 |
| Dioxide | |
| Sequence 5 | |
| Mica | 4.02 |
| Fragrance | 0.3 |

Mix sequence 2 into sequence 1 in a steam bath. Add sequence 3 and mix until uniform. Add sequence 4 and mix until uniform. Add sequence 5 and mix until uniform. Add fragrance and pigments.

### EXAMPLE 3

A long wear lip-gloss was made as follows:

| Compound | w/w/% |
|---|---|
| Phenyl Trimethicone | 12 |
| Isododecane | 9 |
| Polyglyceryl-4 Isostearate, Cetyl | 29.65 |
| PEG/PPG-10/1 Dimethicone, Hexyl Laurate | |
| Silica Silylate | 3.5 |
| Styrene/Acrylates Copolymer | 15 |
| PPG-51/SMDI Copolymer | 8 |
| Trimethylsiloxyphenyl dimethicone | 10 |
| Cera microcristallina | 2.5 |
| 1,2 Hexanediol, caprylyl glycol | 2 |
| Organic or Inorganic Dry Color pigments | 2.75 |
| Mica, Titanium Dioxide | 4.75 |
| Mica | 0.52 |
| Polyglyceryl-4 isostearate, cetyl PEG/PPG- | 2.0 |
| 10/1 Dimethicone, Hexyl Laurate | |
| Fragrance | 0.4 |

The first three ingredients are mixed and heated. Silica Silylate is added to the heated mixture. The polymer is added to the Silica mixture, continue mixing until a homogenous mixture is obtained. Add the next three ingredients and mix to a homogeneous mixture. Add preservatives. Cool and pigments and fragrance.

### EXAMPLE 4

A long wear lip-gloss was made as follows:

| Compound | w/w/% |
|---|---|
| Sequence 1 | |
| Styrene Acrylic Copolymer | 10 |
| Polysilicone 6 | 8.5 |
| Isododecane | 10 |
| Phenyl Trimethicone | 10 |
| Polyglyceryl-4 Isostearate, Cetyl | 7.5 |
| PEG/PPG-10/1 Dimethicone, Hexyl Laurate | |
| Sequence 2 | |
| Silica silylate | 24.5 |
| PPG-51/SMDI Copolymer | 10 |
| Trimethylsiloxyphenyl dimethicone | 10 |
| Microcrystalline rheology agent | 2.0 |
| 1,2 Hexanediol, caprylyl glycol | 0.5 |
| Sequence 3 | |
| Orange 5 | 0.05 |
| Organic or Inorganic Dry Color pigments | 1.48 |
| Sequence 4 | |
| Mica, Titanium Dioxide | 0.5 |
| Calcium Sodium Borosilicate Titanium | 0.5 |
| Dioxide | |
| Sequence 5 | |
| Mica | 4.21 |
| Fragrance | 0.3 |

Mix sequence 2 into sequence 1 in a steam bath. Add sequence 3 and mix until uniform. Add sequence 4 and mix until uniform. Add sequence 5 and mix until uniform. Add fragrance.

### EXAMPLE 5

A long wear lip-gloss was made as follows:

| Compound | w/w/% |
|---|---|
| Sequence 1 | |
| Styrene Acrylic Copolymer | 15 |
| Phenyl Trimethicone | 13 |
| Isododecane | 10 |
| Polyglyceryl-4 Isostearate, Cetyl | 28.4 |
| PEG/PPG-10/1 Dimethicone, Hexyl Laurate | |
| Sequence 2 | |
| Bisdigglyceryl Polyacyladipate-2 | 1.3 |
| PPG-51/SMDI Copolymer | 10 |
| Trimethylsiloxyphenyl dimethicone | 10 |
| Microcrystalline rheology agent | 2.0 |
| 1,2 Hexanediol, caprylyl glycol | 0.5 |
| Sequence 3 | |
| Orange 5 | 0.02 |
| Organic or Inorganic Dry Color pigments | 1.5 |
| Sequence 4 | |
| Mica, Titanium Dioxide | 0.5 |
| Calcium Sodium Borosilicate Titanium | 0.6 |
| Dioxide | |
| Sequence 5 | |
| Silica Silylate | 2.5 |
| Mica | 4.4 |
| Fragrance | 0.3 |

Mix sequence 2 into sequence 1 in a steam bath. Add sequence 3 and mix until uniform. Add sequence 4 and mix until uniform. Add sequence 5 and mix until uniform. Add fragrance.

### EXAMPLE 6

Long wear mascara

w/w %

| Ingredient | 1 | 2 | 3 |
|---|---|---|---|
| Styrene acrylic copolymer | 33.5 | 30.6 | 30.0 |
| Isododecane | 0 | 51.6 | 32.5 |
| Trioctyldodecylcitrate | 6.8 | 7.4 | 7.5 |
| Sunshine Superdeep blue, iron | 0 | 0.5 | 0 |
| blue, TiO2, mica | | | |
| Black iron oxide 33-134 | 6.8 | 7.4 | 7.5 |
| C8-9 isoparaffin | 52.4 | 0 | 0 |
| 12-Hydroxystearic acid | 0 | 2.0 | 0 |
| Water | 0 | 0 | 20 |
| Laureth-4 | 0 | 0 | 1.0 |
| Octyldodeceth-20 | 0 | 0 | 1.0 |
| 1,2 Hexanediol, caprylyl glycol | 0.5 | 0 | 0 |
| Benzoic acid | 0 | 0.1 | 0.1 |
| Methyl paraben | 0 | 0.3 | 0.3 |
| Propyl paraben | 0 | 0.1 | 0.1 |
| BHT | 0 | 0.1 | 0.1 |

The gloss and comfort were evaluated in a 5 day monadic claims study on 50 consumers comparing the formula of the present invention an existing marketed formula.

The composition had good cosmetic properties as for example feel and appearance. Additionally, the product provided acceptable wear for up to 8 hours

Consumers were given a sample of the lip-gloss and asked to apply as needed for four days. On the fifth day the consumers were given a set of questions and asked to apply the lip-gloss at 2, 4, 6, 8 and 10 hours and to answer the questions at each time point, the results are presented above.

What is claimed is:

1. A cosmetic composition comprising, a styrene/acrylate copolymer, at least one siloxane, at least one rheology agent, and at least one ester.

2. The composition of claim 1, wherein the composition may contain a hydrocarbon.

3. The composition of claim 1, wherein the composition contains at least one particulate phase.

4. The composition of claim 1, wherein the composition contains at least one additive.

5. The composition of claim 1, wherein the composition contains at least one pigment.

6. The composition of claim 1, wherein the styrene/acrylate copolymer comprises, a tert-butylstyrene/C₁-C₁₀ alkyl acrylate/methacrylate copolymer.

7. The composition of claim 1, wherein the siloxane comprises phenyl trimethicone, polysilicone-6, trimethylsiloxyphenyl dimethicone and mixtures thereof.

8. The composition of claim 1, wherein the ester comprises polyglyceryl-4 isostearate, hexyl laurate, trioctyldodecylcitrate and mixtures thereof.

9. The composition of claim 1, wherein the rheology agent comprises a wax.

11. A cosmetic composition comprising, by weight of the total composition:
a. about 3-20% of a styrene/acrylate copolymer;
b. about 0.5-5% of a rheology agent;
c. about 8-20% of a siloxane; and
d. about 5-30% of an ester.

12. The composition of claim 1, wherein the composition is a lip-gloss.

13. The composition of claim 1, wherein the composition is a mascara.

14. The composition of claim 2, wherein the hydrocarbon comprises isododecane, isoparaffin, cyclomethicone, linear methicone, and mixtures thereof.

15. The composition of claim 2, wherein the hydrocarbon comprises about 3-40% by weight of the total composition.

16. The composition of claim 11, wherein the composition may contain a hydrocarbon.

17. The composition of claim 11, wherein the composition contains at least one particulate phase.

18. The composition of claim 11, wherein the composition contains at least one additive.

19. The composition of claim 11, wherein the composition contains at least one pigment.

20. The composition of claim 12, wherein the composition retains gloss properties, comfort without irritation and staying power for up to about 8 hours.
